(19) United States
(12) Patent Application Publication
(10) Pub. No.: US 2025/0290912 A1
(43) Pub. Date: Sep. 18, 2025

(54) INTELLIGENT MOTORIZED WATER-TESTING DRONE ATTACHMENT

(71) Applicants: Michael Matthew Wilkinson, San Carlos, CA (US); Jaiveer Mehta, San Carlos, CA (US); Anthony Jonathan Wilkinson, San Carlos, CA (US); Ishan Mookerjee, San Mateo, CA (US)

(72) Inventors: Michael Matthew Wilkinson, San Carlos, CA (US); Jaiveer Mehta, San Carlos, CA (US); Anthony Jonathan Wilkinson, San Carlos, CA (US); Ishan Mookerjee, San Mateo, CA (US)

(21) Appl. No.: 19/077,666

(22) Filed: Mar. 12, 2025

Related U.S. Application Data
(60) Provisional application No. 63/564,982, filed on Mar. 13, 2024.

(51) Int. Cl. G01N 33/18; B64U 20/80; B64U 101/35

(52) U.S. Cl. CPC G01N 33/1886; B64U 20/80; B64U 2101/35

(57) ABSTRACT

This disclosure provides systems, devices, apparatus, and methods, including computer programs encoded on storage media, for water testing. A drone attachment detects a suitable testing region in a body of water using one or more sensors. The drone attachment deploys a test strip into the suitable testing region in the body of water. The drone attachment performs a water testing while the test strip is inside the body of water. The drone attachment retracts the test strip from the body of water after a testing period is over.

### CROSS-REFERENCE TO RELATED APPLICATIONS

This application claims benefit of provisional U.S. Patent Application No. 63/564,982, filed Mar. 13, 2024, which is incorporated herein by reference in its entirety.

### TECHNICAL FIELD

The present disclosure relates generally to water testing, and more particularly, to a drone attachment for water testing.

### BACKGROUND

Ocean acidification is a phenomenon caused by the absorption of atmospheric carbon dioxide (CO2) by the Earth's oceans. Approximately 30% of greenhouse gas emissions are absorbed by the ocean. As the climate crisis continues, an unexpected byproduct of global warming is making itself apparent. As CO₂ levels rise in water, PH levels are decreasing and salinity levels are increasing, resulting in the phenomenon of ocean acidification. As the acidity of the oceans rises, it is harder for creatures to survive. Ocean acidification can negatively affect and disrupt marine food webs and ecosystems.

While marine biologists research on how to combat ocean acidification, water sampling and testing is important in monitoring and studying ocean acidification. However, water testing and sampling is a long and time consuming process.

### BRIEF SUMMARY

The following presents a simplified summary of one or more aspects in order to provide a basic understanding of such aspects. This summary is not an extensive overview of all contemplated aspects. This summary neither identifies key or critical elements of all aspects nor delineates the scope of any or all aspects. Its sole purpose is to present some concepts of one or more aspects in a simplified form as a prelude to the more detailed description that is presented later.

Water sampling and testing is valuable in monitoring and studying ocean acidification. One way to perform water sampling is using smart drones. Currently, the majority of water sampling drones are using containers attached to the drones to collect water samples. Some of the water sampling drones are water-proof and enter the waters with attached containers. Some water sampling drones lower water bottles into a body of water to collect water samples. However, these water sampling drones with attached containers requires operators to manually control the operation, collect and store the water samples. The water samples will be used for manually testing at a later time. It will take some time to have the testing results. There is also a risk of contamination during transportation. There is a need to develop an automatic water testing process with high efficiency while saving resources.

Aspects of the present disclosure address the above-noted and other deficiencies by a drone attachment which can intelligently detect a suitable region in a body of water, automatically deploy a testing strip on command, perform water sampling and testing while the testing strip is in the water, and retract the testing strip after the testing period is over, in order to provide environmental organizations to help determine areas that have been affected by ocean acidification. The drone attachment is capable of perform automatic color recognition of suitable regions, remotely deploying a water-testing strip, conducting water tests, and retracting the strip afterward. The automatic color detection system and automatic deployment/retraction system solves the problem of requiring a manned operator to watch the camera feed and manually input commands. In this way, the efficiency of the water testing system is significantly improved while saving resources. Another advantage of this system is allowing water testing at a much greater range than the remote control.

According to some aspects, a drone attachment detects a suitable testing region in a body of water using one or more sensors. The drone attachment deploys a test strip into the suitable testing region in the body of water. The drone attachment performs a water testing while the test strip is inside the body of water. The drone attachment retracts the test strip from the body of water after a testing period is over.

According to some aspects, a drone attachment comprises one or more sensors, a deployment/retraction system, and a processor. The deployment/retraction system includes a motor with a motor rod, and the motor rod is configured to be connected to a string connected with one or more test strips. The processor configured to: detect a suitable testing region in a body of water using the one or more sensors; deploy the one or more test strips into the suitable testing region in the body of water; perform a water testing while the test strip is inside the body of water; and retracting the test strip from the body of water after a testing period is over.

According to some aspects, a drone with a drone attachment comprises a drone main body and a drone attachment attached to the drone main body. The drone attachment comprises one or more sensors, a deployment/retraction system, and a processor. The deployment/retraction system includes a motor with a motor rod, and the motor rod is configured to be connected to a string connected with one or more test strips. The processor configured to: detect a suitable testing region in a body of water using the one or more sensors; deploy the one or more test strips into the suitable testing region in the body of water; perform a water testing while the test strip is inside the body of water; and retracting the test strip from the body of water after a testing period is over.

By using the drone attachment for remote water testing, high efficiency and conservation of manpower and funds may be achieved. Test carries out automatically, thus enabling labor saving through automation. On-site testing protects from contamination of the water sample during transportation. This technique also enables real time in-flight test results analysis and transmission for fast test results. In cooperation with environmental organizations, this attachment could allow several speedy trials of testing in various locations with minimal equipment and resources.

### BRIEF DESCRIPTION OF THE DRAWINGS

FIG. 1A and FIG. 1B illustrate block diagrams of a top view and a side view of a drone attachment respectively according to an embodiment.

FIG. 2A illustrates a drone with a drone attachment detecting a suitable region in a body of water according to an embodiment.

FIG. 2B illustrates a drone with a drone attachment deploying a testing strip in a body of water according to an embodiment.

FIG. 3A and FIG. 3B illustrates examples of a drone with a drone attachment having a plurality of test strips according to an embodiment.

FIG. 4 is a flow diagram illustrating drone-based water-testing using a drone attachment according to an embodiment.

FIG. 5 is a flowchart illustrating an example of automatic color detection, test strip deployment/retraction for water testing according to an embodiment.

FIG. 6 illustrates an example of a test strip for water testing according to an embodiment.

FIG. 7 is a flowchart illustrating a process of automatic water testing results analysis according to an embodiment.

FIG. 8 is a flowchart of a method of drone-based water testing according to an embodiment.

### DETAILED DESCRIPTION

Various embodiments and aspects of the disclosures will be described with reference to details discussed below, and the accompanying drawings will illustrate the various embodiments. The following description and drawings are illustrative of the disclosure and are not to be construed as limiting the disclosure. Numerous specific details are described to provide a thorough understanding of various embodiments of the present disclosure.

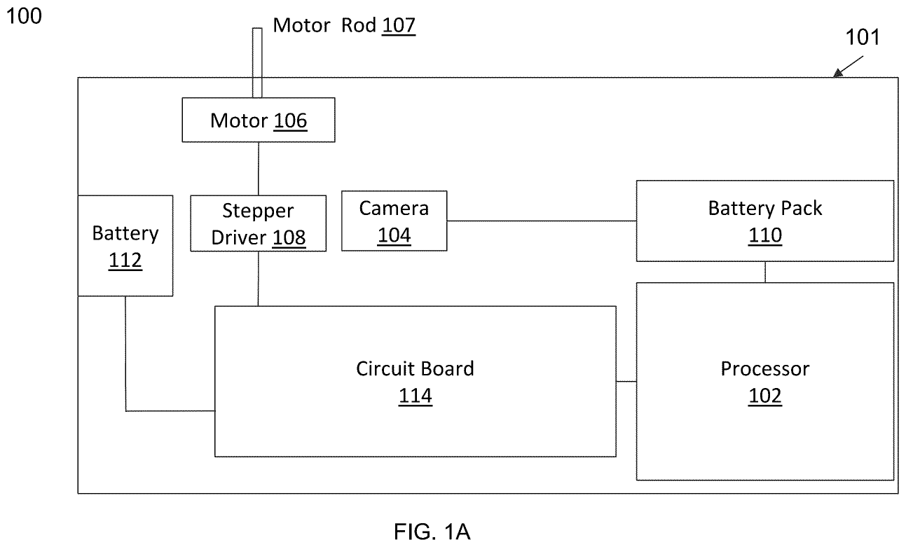

FIG. 1A and FIG. 1B illustrate block diagrams of a top view and a side view of a drone attachment 101 respectively according to an embodiment. The drone attachment 101 is capable of perform automatic color recognition of suitable regions, remotely deploying a water-testing strip, conducting water tests, and retracting the strip afterward.

Referring to FIG. 1A, the drone attachment 101 includes a processor 102, one or more sensors (e.g., a camera 104), a deployment/retraction system (e.g., a motor 106, a stepper driver 108, a circuit board 114), and power supply (e.g., battery 112). The drone attachment 101 may also include battery pack 110. The processor 102 is connected with the camera 104, the motor 106 and the circuit board 114. The processor 102 is configured to perform automatic color detection and automatic deployment/retraction of a testing strip (not shown). In some examples, the processor 102 is further configured to automatically analyze the test results of the test strip, and transmit the test results of the test strip to a remote user.

In some examples, the drone attachment 101 may be attached to a drone. In some examples, the drone attachment 101 may be attached to a flying object, such as an unmanned flying object.

Referring to FIG. 1B, in some examples, the drone attachment 101 includes a housing 101a. The processor 120, the motor 106, the stepper driver 108, the circuit board 114, the battery 112, and the battery pack 110 are disposed inside the housing 101a. The camera 104 is mounted at a bottom of the housing 101a. The motor 106 is mounted at a side of the housing 101a. The motor 106 may have a motor rod 107, which is extending out of the housing 101a. The motor rob is configured to be connected with a string, which is connected with a test strip.

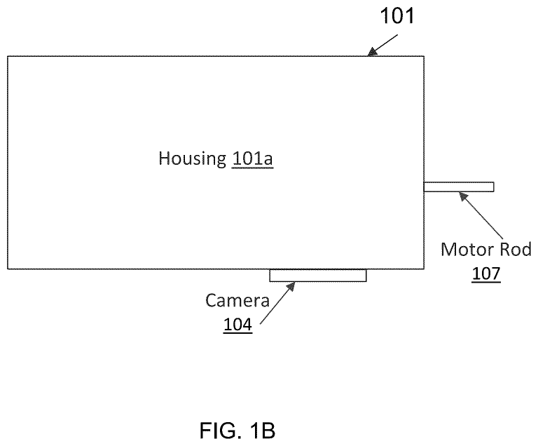

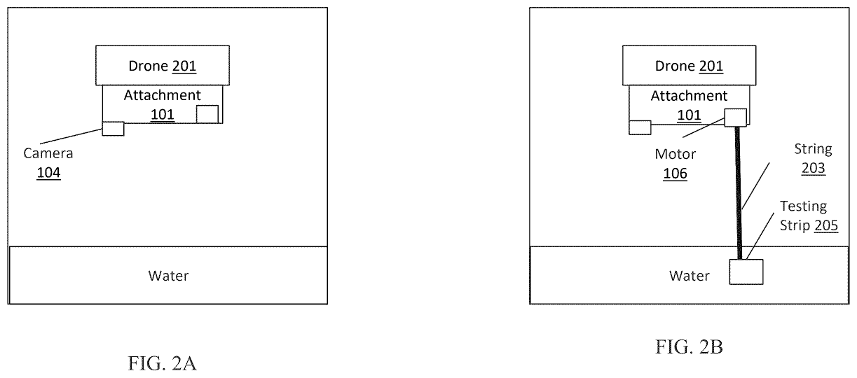

FIG. 2A illustrates a drone 201 with the drone attachment 101 detecting a suitable region in a body of water according to an embodiment. The drone 201 with the drone attachment 101 automatically detects a suitable region in a body of water by using the color detecting system. The suitable region in a body of water may refer to a region without obstacles, rocks, or sea animals, thus being suitable for perform testing. There is no obstacle obstructing the testing process in the suitable region. The video from the camera 104 are input into the processor 102. The processor 102 is configured to detect the suitable region based on the color detecting system. To solve the issue of false triggers, a false-trigger checking module is developed. In some examples, the color blue needs to be detect for a certain number of times consecutively. Once the color blue is detected, the motor isn't triggered unless the false-trigger checking module approves the detection.

FIG. 2B illustrates the drone 201 with the drone attachment 101 deploying a testing strip 205 in a body of water according to an embodiment. After detecting the suitable region in the body of water, the processor 102 is configured to send command to the motor 106 to automatically deploy the testing strip 205 by rotating the motor 107 in a first direction. The testing strip 205 is connected to the motor rod through a string 203. The testing strip 205 is deployed into the water by rotating the motor 107 in a first direction to unwind the string 203. After the testing strip 205 is deployed into the water, the water testing is performed while the testing strip is in the water. After a testing period is over, the processor 102 is configured to send command to the motor 106 to automatically retract the testing strip 205 from the water by rotating the motor 107 in a second direction to winding the string 203. Thus, the testing strip is automatically retracted after the testing period is over.

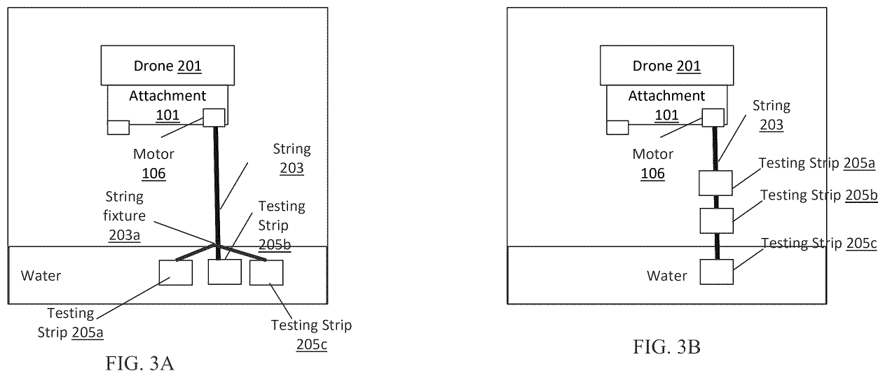

FIG. 3A and FIG. 3B illustrates examples of a drone with a drone attachment having a plurality of test strips according to an embodiment. In some examples, the drone attachment 101 may carry a plurality of test strips (e.g., 205a, 205b, and 205c). The plurality of test strips may be connected to the motor 106 through the string 203. In this way, a plurality of water tests may be performed, simultaneously or sequentially, with the deployment of the string 203. The plurality of water tests may be performed for multiple purposes, e.g., testing multiple water qualities. In some examples, the plurality of water tests may be performed for redundancy purpose. In some examples, the plurality of test strips (e.g., 205a, 205b, and 205c) may be connected to the same string 203. In some examples, the plurality of test strips (e.g., 205a, 205b, and 205c) may be connected to a plurality of strings. The drone with the drone attachment may fight to a plurality of locations to perform a plurality of water tests using the plurality of test strips.

Referring to FIG. 3A, in some examples, the string 203 may include a fixture 203a, which may be connected to the plurality of test strips (e.g., 205a, 205b, and 205c). The plurality of water tests may be performed simultaneously with one deployment of the string 203.

Referring to FIG. 3B, in some examples, the plurality of test strips (e.g., 205a, 205b, and 205c) may be connected to the string 203 in series. A first test strip 205a may be deployed first to conduct a first water test. After the first water test period is over, a second test strip 205b may be deployed to conduct a second water test, and so on. The plurality of water tests may be performed sequentially with one deployment of the string 203. The sequential water tests may be performed for different water tests with different testing periods.

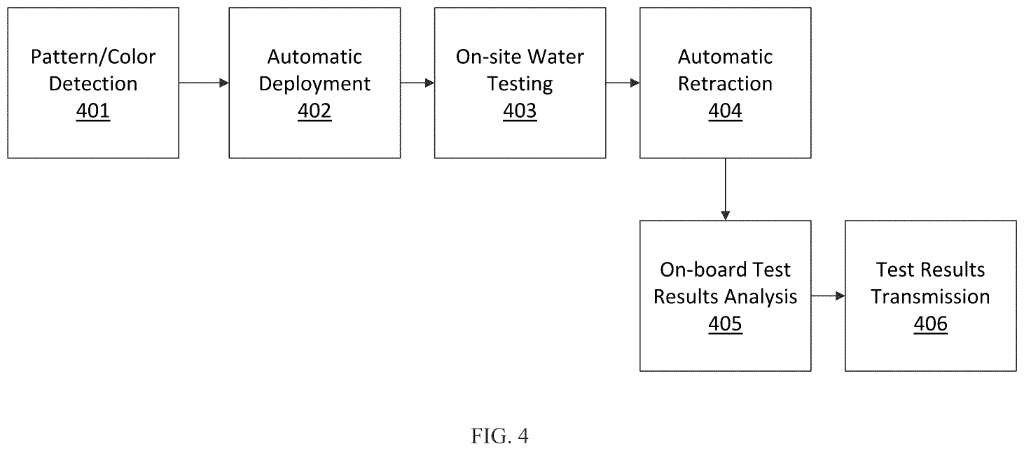

FIG. 4 is a flow diagram illustrating drone-based water-testing using the drone attachment 101 according to an embodiment. In some examples, the drone 201 with the drone attachment 101 may fly to a remote area. The one or more sensors (e.g., a camera 104) may detect the environment, and perform pattern recognition or color recognition 401. After detecting a suitable area for testing in a body of water, the deployment system (e.g., a motor 106, a stepper driver 108, a circuit board 114) may be activated and perform automatic deployment 402 of one or more test strips. After the one or more test strips being deployed inside the water, on-site water testing 403, including one or more water tests using the one or more test strips, is performed. After the testing period is over, the retraction system may perform automatic retraction 404 of the one or more test strips.

In some examples, the processor 102 may be configured to automatically analyze the test results of the test strip. After the one or more test strips being retracted from the water, one or more images of the one or more test strips, after conducting the water test, are being input to the processor. The one or more images of the one or more test strips may be captured by the camera 104, or may be captured by other cameras disposed in the drone attachment 101. In this way, on-board test results analysis 405 may be performed. The processor 102 may be configured to automatically obtain or analyze the test results of the one or more test strips by pattern recognition or color recognition. Afterwards, the test results of the one or more test strips may be transmitted to the remote user or operator. The test results transmission 406 may be performed by using wireless communication.

In some examples, the processor 102 may include a pattern/color recognition module 401, an automatic deployment module 402, an on-site testing module 403, an automatic retraction module 404, an on-board test results analysis module 405, and a test results transmission module 406. The processor 102 may be configured to perform the pattern/color recognition 401, the automatic deployment module 402, the on-site testing 403, the automatic retraction 404, the on-board test results analysis 405, and the test results transmission 406.

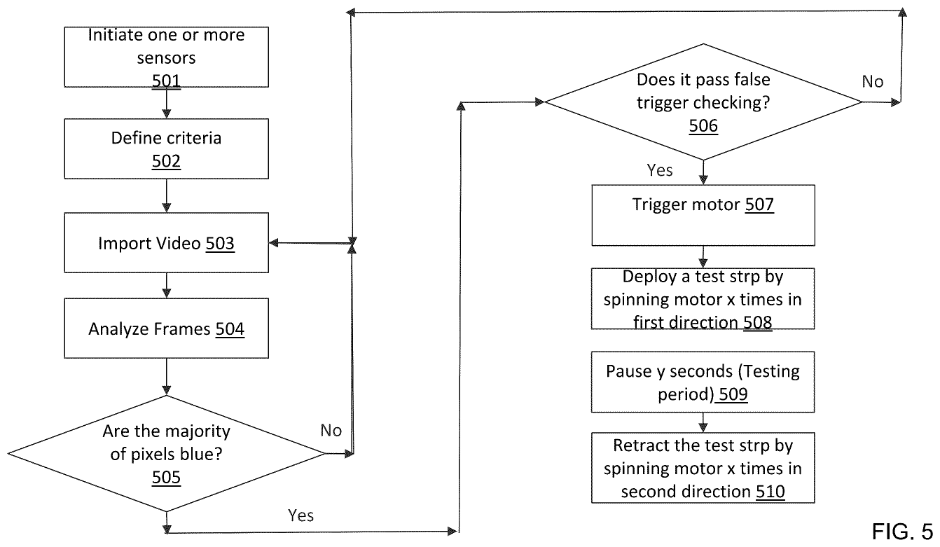

FIG. 5 is a flowchart illustrating an example of automatic color detection, test strip deployment/retraction for water testing according to an embodiment. At first, the one or more sensor (e.g., the camera 104) are initiated 501. Then, criteria for pattern recognition or color recognition is defined 502. As an example, color recognition may be used to detect a suitable region/area for water testing in a body of water. The color “blue” may be defined as the criteria for color recognition of the suitable region/area for water testing. The suitable region/area for water testing may refer to a region/area within the body of water that is free of obstacles, sea plants, sea animals, rocks, or trashes.

The videos of the one or more sensor (e.g., the camera) may be inputted/imported 503 into the processor 102. The processor 102 may analyze 504 the frames of the videos of the one or more sensor (e.g., the camera).

Then, pattern recognition or color recognition may be used to determine the suitable region/area for water testing. In one example, whether the majority of the pixels are blue is determined 505. If the majority of the pixels are blue, whether the detection passes a false trigger checking 506 is determined. In some example, the color detection/pattern detection system may be too sensitive, resulting false triggers/detections. In order to prevent false triggers, the false trigger checking is developed. As an example, the color “blue” needs to be detected for a certain number of times consecutively, to pass the false trigger checking. By using the false trigger checking, once blue is detected, the motor isn't triggered unless the false trigger checking approves the detection.

If the detection passes the false trigger checking, the deployment system is activated. The motor is triggered 507. To deploy 508 the string connected with the one or more test strips, the motor spins a plurality of times in a first direction. The first direction may be clockwise or counter-clockwise.

After the one or more test strips are deployed into the water, a timer starts. The motor may pause 509 for a time duration of a testing period. The on-site water testing may be performed while the one or more test strips are inside the water,

After the testing period is over, the timer expires. The retraction system is activated. To retract 510 the string connected with the one or more test strips, the motor spins a plurality of times in a second direction. The second direction is opposite to the first direction. If the first direction is clockwise, the second direction is counter-clockwise. If the first direction is counter-clockwise, the second direction is clockwise.

In some examples, machine learning may be used to perform pattern recognition to identify the suitable region/area for water testing.

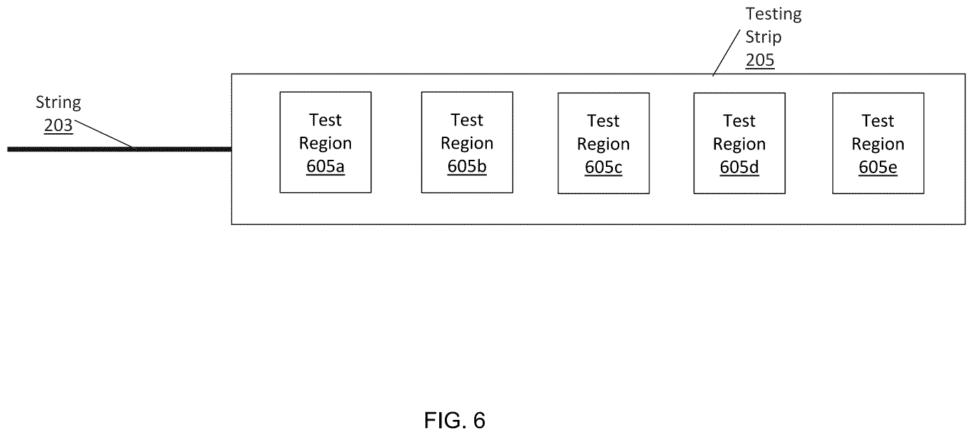

FIG. 6 illustrates an example of a test strip 205 for water testing according to an embodiment. In some examples, the test strip may include multiple test regions (e.g., 605a, 605b, 605c, 605d, and 605c). In each of the multiple test regions, different materials to disposed to test different properties of the water. By analyzing the patterns or colors in multiple test regions in the test strip 205, the test results of multiple properties of the water may be obtained. For example, the multiple test regions of the testing strip shows different colors with different numbers which may indicate the levels of pH, general hardness, carbonate hardness, nitrate content, and nitrite content.

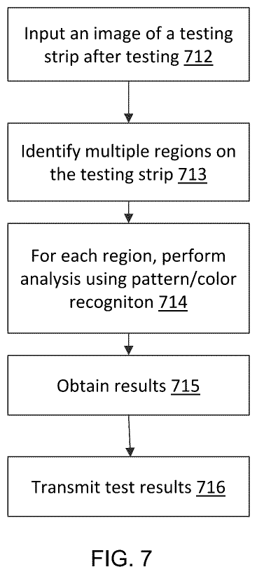

FIG. 7 is a flowchart illustrating a process of automatic water testing results analysis according to an embodiment. In some examples, the camera 104 or a second camera in the drone attachment 101 may be used to capture an image of a test strip after the testing is over, e.g., after the test strip being retracted from the water. The processor 102 may include the on-board test results analysis module 405 to obtain test results timely by performing test results analysis of the test strip.

Referring to FIG. 7, the image of the test strip may be inputted 712 into the on-board test results analysis module 405 of the processor 102. A plurality of test regions on the test strip may be identified 713 by using the on-board test results analysis module 405. Using the camera mounted on the drone attachment, photos of the test strip can be taken instantly and the results can be relayed back to the operator.

For each of the plurality of test regions, analysis of test results may be performed 714 using color recognition or pattern recognition. In some examples, color recognition may be used to analyze the test results in each of the plurality of test regions on the test strip. For example, for some test strips, the water qualities may be indicated by the color change in each of the plurality of test regions after the water testing. In such situations, the test results may be automatically obtained by using color recognition algorithm.

In some examples, pattern recognition may be used to analyze the test results in each of the plurality of test regions on the test strip. For example, for some test strips, the water qualities may be indicated by a pattern change in each of the plurality of test regions after the water testing. In such situations, the test results may be automatically obtained by using pattern recognition algorithm or using machine learning.

The test results of water qualities may be obtained 715 based on the analysis of test results using color recognition or pattern recognition. The test results of water qualities may be transmitted to a remote user or operator wirelessly.

In this way, the test results of water qualities in a remote may be obtained in real time, with high efficiency, saving human labor and resources. Therefore, the efficiency of the water testing system is significantly improved while saving resources. Another advantage of this system is allowing water testing at a much greater range than the remote control range.

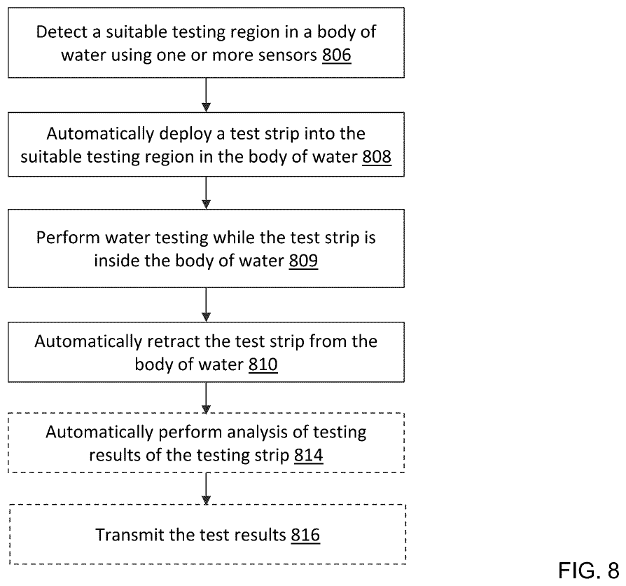

FIG. 8 is a flowchart of a method of drone-based water testing according to an embodiment. The method may be performed by a drone attachment, such as the drone attachment 101.

In some embodiments, the drone attachment detects 806 a suitable testing region in a body of water. For example, the drone attachment detects 806 the suitable testing region in the body of water by using automatic color detection or pattern detection, and combined with false trigger checking.

After detecting the suitable testing region, the drone attachment automatically deploys 808 a test strip into the suitable testing region in the body of water. For example, the drone attachment automatically deploys 808 the test strip by deploy/unwind a string connected with the test strip. The drone attachment automatically activates 808 the motor for deployment of the testing strip into the suitable region based on the results of color recognition or pattern recognition.

While the test strip is inside the body of water, the drone attachment performs 809 water testing. After a testing period is over, the drone attachment automatically retracts 810 the test strip from the body of water.

In some examples, the drone attachment may automatically perform 814 analysis of testing results of the testing strip. For example, the drone attachment automatically performs 814 analysis of testing results of the testing strip based on the image of the test strip after the testing using pattern recognition or color recognition. The drone attachment may transmit 816 the testing results of the testing strip to a remote operator or user.

By using this method, high efficiency and conservation of manpower and funds may be achieved. On-site testing protects from contamination of the water sample during transportation. The method enables real time in-flight test results analysis and transmission for fast test results. In cooperation with environmental organizations, the method may allow several speedy trials of testing in various locations with minimal equipment and resources.

The detailed description set forth herein describes various configurations in connection with the drawings and does not represent the only configurations in which the concepts described herein may be practiced. The detailed description includes specific details for the purpose of providing a thorough explanation of various concepts. However, these concepts may be practiced without these specific details. In some instances, well known structures and components are shown in block diagram form in order to avoid obscuring such concepts.

If the functionality described herein is implemented in software, the functions may be stored on, or encoded as, one or more instructions or code on a computer-readable medium, such as a non-transitory computer-readable storage medium. Computer-readable media includes computer storage media and can include a random-access memory (RAM), a read-only memory (ROM), an electrically erasable programmable ROM (EEPROM), optical disk storage, magnetic disk storage, other magnetic storage devices, combinations of these types of computer-readable media, or any other medium that can be used to store computer executable code in the form of instructions or data structures that can be accessed by a computer. Storage media may be any available media that can be accessed by a computer.

Aspects, implementations, and/or use cases described herein may be implemented across many differing platform types, devices, systems, shapes, sizes, and packaging arrangements. For example, the aspects, implementations, and/or use cases may come about via integrated chip implementations and other non-module-component based devices, such as end-user devices, vehicles, communication devices, computing devices, industrial equipment, retail/purchasing devices, medical devices, artificial intelligence (AI)-enabled devices, machine learning (ML)-enabled devices, etc. The aspects, implementations, and/or use cases may range from chip-level or modular components to non-modular or non-chip-level implementations, and further to aggregate, distributed, or original equipment manufacturer (OEM) devices or systems incorporating one or more techniques described herein.

The description herein is provided to enable a person skilled in the art to practice the various aspects described herein. Various modifications to these aspects will be readily apparent to those skilled in the art, and the generic principles defined herein may be applied to other aspects. Thus, the claims are not limited to the aspects described herein, but are to be interpreted in view of the full scope of the present disclosure consistent with the language of the claims.

Reference to an element in the singular does not mean “one and only one” unless specifically stated, but rather “one or more.” Terms such as “if,” “when,” and “while” do not imply an immediate temporal relationship or reaction. That is, these phrases, e.g., “when,” do not imply an immediate action in response to or during the occurrence of an action, but simply imply that if a condition is met then an action will occur, but without requiring a specific or immediate time constraint for the action to occur. The terms “may”, “might”, and “can”, as used in this disclosure, often carry certain connotations. For example, “may” refers to a permissible feature that may or may not occur, “might” refers to a feature that probably occurs, and “can” refers to a capability (e.g., capable of). The phrase “For example” often carries a similar connotation to “may” and, therefore, “may” is sometimes excluded from sentences that include “for example” or other similar phrases.

Unless specifically stated otherwise, the term “some” refers to one or more. Combinations such as “at least one of A, B, or C” or “one or more of A, B, or C” include any combination of A, B, and/or C, such as A and B, A and C, B and C, or A and B and C, and may include multiples of A, multiples of B, and/or multiples of C, or may include A only, B only, or C only. Sets should be interpreted as a set of elements where the elements number one or more. Terms or articles such as “a”, “an”, and/or “the” may refer to one of an item, feature, element, etc., that the term or article precedes, or may refer to more than one of said item, feature, element, etc. that the term or article precedes. For example, the recitation “a widget” does not preclude reference to multiples of said widget, as “multiple widgets” necessarily includes “a widget”. Hence, the recitation “a widget” may be interpreted as “at least one widget” or, similarly, interpreted as “one or more widgets”.

Unless otherwise specifically indicated, ordinal terms such as “first” and “second” do not necessarily imply an order in time, sequence, numerical value, etc., but are used to distinguish between different instances of a term or phrase that follows each ordinal term.

Reference numbers, as used in the specification and figures, are sometimes cross-referenced among drawings to denote same or similar features. A feature that is exactly the same in multiple drawings may be labeled with the same reference number in the multiple drawings. A feature that is similar among the multiple drawings, but not exactly the same, may be labeled with reference numbers that have different leading numbers but have one or more of the same trailing numbers (e.g., 206, 306, 406, etc., may refer to similar features in the drawings). Hence, like numbers may refer to like actions.

Structural and functional equivalents to elements of the various aspects described throughout this disclosure that are known or later come to be known to those of ordinary skill in the art are expressly incorporated herein by reference and are encompassed by the claims. The words “module,” “mechanism,” “element,” “device,” and the like may not be a substitute for the word “means.” As such, no claim element is to be construed as a means plus function unless the element is expressly recited using the phrase “means for.” As used herein, the phrase “based on” shall not be construed as a reference to a closed set of information, one or more conditions, one or more factors, or the like. In other words, the phrase “based on A”, where “A” may be information, a condition, a factor, or the like, shall be construed as “based at least on A” unless specifically recited differently.

What is claimed is:

1. A method of drone-based water testing, comprising:
detecting, by a processor, a suitable testing region in a body of water;
deploying, by the processor, a test strip into the suitable testing region in the body of water;
performing, by the processor, a water testing while the test strip is inside the body of water; and
retracting, by the processor, the test strip from the body of water after a testing period is over.

2. The method of claim 1, further comprising:
performing, by the processor, analysis of testing results of the testing strip.

3. The method of claim 2, further comprising:
transmitting, by the processor, the testing results to a remote user wirelessly.

4. A drone attachment, comprising:
one or more sensors;
a deployment/retraction system including a motor with a motor rod, the motor rod is configured to be connected to a string connected with one or more test strips; and
a processor configured to:
detect a suitable testing region in a body of water using the one or more sensors;
deploy the one or more test strips into the suitable testing region in the body of water;
perform a water testing while the test strip is inside the body of water; and
retracting the test strip from the body of water after a testing period is over.

5. The drone attachment of claim 4, wherein the one or more sensors are configured to capture an image of the test strip after being retracted from the water, wherein the processor is further configured to:
perform analysis of testing results of the testing strip.

6. The drone attachment of claim 5, wherein the processor is further configured to:
transmit the testing results to a remote user wirelessly.

7. A drone with a drone attachment, comprising:
a drone main body; and
a drone attachment attached to the drone main body, the drone attachment comprising:
one or more sensors;
a deployment/retraction system including a motor 106 with a motor rod, the motor rod is configured to be connected to a string connected with one or more test strips; and
a processor configured to:
detect a suitable testing region in a body of water using the one or more sensors;
deploy the one or more test strips into the suitable testing region in the body of water;
perform a water testing while the test strip is inside the body of water; and
retracting the test strip from the body of water after a testing period is over.

8. The drone with the drone attachment of claim 7, wherein the one or more sensors are configured to capture an image of the test strip after being retracted from the water, wherein the processor is further configured to:
perform analysis of testing results of the testing strip.

9. The drone with the drone attachment of claim 8, wherein the processor is further configured to:
transmit the testing results to a remote user wirelessly.
